(19) United States
(12) Patent Application Publication
(10) Pub. No.: US 2004/0037856 A1
(43) Pub. Date: Feb. 26, 2004

(54) Use of compounds containing a thio-ether, sulphoxide or sulphone function as cosmetic anti-pollution agent

(76) Inventors: Philippe Catroux, Nogent-sur-Marne (FR); Jean Maignan, Tremblay-en-France (FR); Jose Cotovio, Danmartin-en-Goele (FR)

Correspondence Address: D Douglas Price, Steptoe & Johnson, 1330 Connecticut Avenue NW, Washington, DC, 20036, US

(21) Appl. No.: 10/276,541

(22) PCT Filed: May 14, 2001
(86) PCT No.: PCT/FR01/01453

(30) Foreign Application Priority Data
May 18, 2000 (FR) 00/06386

(51) Int. Cl. A61K 7/00

(52) U.S. Cl. 424/401

(57) ABSTRACT

The invention concerns the use for topical application of compounds containing a thio-ether, sulphoxide or sulphone function as cosmetic anti-pollution agent and a cosmetic treatment method to obtain protection of the organism against pollution effects.

The present invention relates essentially to a novel use of compounds containing a thioether, sulfoxide or sulfone function, as antipollution cosmetic agents.

Urban environments are regularly subjected to peaks of pollution. The atmospheric pollutants that are widely represented by the primary and secondary products of combustion represent a major source of environmental oxidative stress.

Urban pollution is composed of various types of chemical products, xenobiotics and particles.

Three major categories of pollutants can exert deleterious effects on the skin and the hair: gases, heavy metals and particles that are combustion residues onto which are adsorbed a large number of organic compounds.

It is the outermost tissues that are initially and directly exposed to environmental toxic agents. The skin is directly and frequently exposed to the prooxidizing environment. The environmental sources of oxidizing agents include oxygen, solar UV radiation and also, in polluted air, ozone, nitrogen oxides and sulfur oxides. The atmospheric pollutants represented by the primary and secondary products of domestic and industrial combustion such as monocyclic and polycyclic aromatic hydrocarbons are also a major source of oxidative stress. The skin is particularly sensitive to the action of oxidative stress and the outermost layer serves as a barrier against oxidative damage. In most circumstances, the oxidizing agent is likely to be neutralized after reaction with keratin materials, but the reaction products formed may be responsible for attacks on cells and tissues.

The stratum corneum, which is the skin's barrier, is the site of contact between the air and skin tissue. The lipid/protein two-phase structure is a crucial factor of this skin barrier function. These elements can react with oxidizing agents and be impaired, which will promote the phenomena of desquamation. Ozone-induced lipid peroxidation can impair the skin in two ways:

1/ the oxidation and degradation of the lipids of the stratum corneum can impair the barrier function of the stratum corneum. Disruption of the outer lipids and of the protein architecture appear to be triggering factors in many dermatoses (psoriasis, atopic dermatitis and irritant dermatitis).

2/ the increased formation of lipid oxidation products in the upper layers of the skin can trigger attacks in the adjacent layers of skin. The reaction of ozone (O₃) with unsaturated lipids involves addition reactions known as the Criegge reaction, according to the scheme described below:

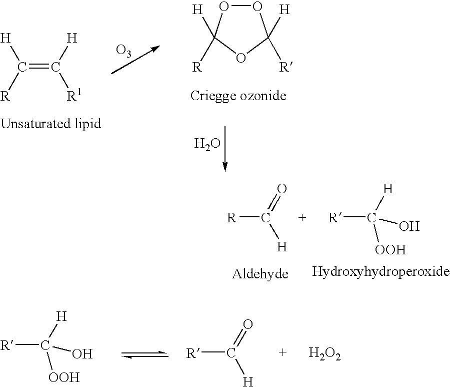

This process leads in a second stage to cleavage of the lipid chains and to the formation of hydroperoxides and aldehydes and also of hydrogen peroxide. This is a specific mechanism that is different than the lipoperoxidation mechanism conventionally described, which is mediated by a radical. The secondary or tertiary lipid oxidation products induced with ozone, which are less reactive than ozone but have a longer lifetime, can propagate the effect of ozone. On account of their relative stability, lipid oxidation and peroxidation products, i.e. cholesterol oxides and aldehydes, have the potential to impair cells at remote sites not directly exposed to ozone.

A significant level of oxidative attack on the surface layers of the stratum can initiate localized subjacent inflammatory processes, leading to the recruitment of phagocytes, which, by generating oxidizing agents, will amplify the initial oxidative processes.

In urban pollution, the concomitant exposure to ozone and to UV can cause a synergistic oxidative stress.

Similarly, it may be thought that there is synergistic action between ozone and combustion-derived organic compounds (monocyclic and polycyclic aromatic compounds).

Solutions have already been envisioned in cosmetic and therapeutic treatments by protecting tissues with compounds with sulfur-containing groups which behave like heavy metal sequestering agents, for instance the metallothioneines in patent EP 0 557 042 A1 and the amino acid compounds with thiol groups in patent application EP 0 914 815 A1.

Moreover, carboxylic acids bearing a thioether function or a sulfoxide function are described in patent EP-A-0 576 287, which claims their capacity to improve the elasticity of the skin.

Prior uses exist for carboxylic acids bearing a sulfur-containing function to promote the desquamation of the skin or to stimulate epidermal renewal.

The problem posed is thus that of protecting the skin against toxic gases, and more particularly ozone, and the deleterious effects of these gases encountered in urban pollution.

It has now been found, entirely surprisingly, that the use in topical application of compounds containing a thioether, sulfoxide or sulfone function makes it possible to protect keratin materials, the skin and integuments against the deleterious effects of gases, and more particularly ozone.

The Applicant has discovered that these compounds make it possible, inexplicably, to preserve and protect keratin materials, the skin and integuments against the harmful effects of pollution.

Thioethers give with ozone stable oxidation products in the form of sulfoxides, while sulfoxides may, in contact with ozone, be converted into the corresponding sulfones. These sulfones, in the presence of an excess of ozone, are converted into the corresponding sulfonic acid.

One subject of the present invention is the use in topical application of compounds containing a thioether, sulfoxide or sulfone function, as antipollution cosmetic agents.

The expression “antipollution cosmetic agent” means an agent that protects the skin and keratin materials so as to prevent, attenuate and/or eliminate the deleterious effects of toxic gases such as ozone and organic compounds that are combustion residues.

These compounds containing a thioether, sulfoxide or sulfone function correspond to formulae I, II and III below:

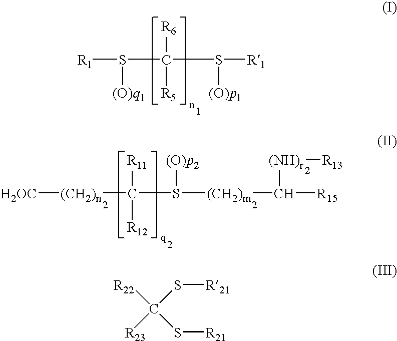

n₁ represents an integer ranging from 1 to 10,

p₁ and q₁, which may be identical or different, represent 0, 1 or 2,

n₂ represents an integer ranging from 0 to 5,

p2 represents 0, 1 or 2,

q₂ represents 0, 1, 2 or 3,

m₂ represents an integer ranging from 0 to 10,

r₂ represents 0 or 1,

q₂ and n₂ never both simultaneously being zero.

The radicals R₁ and R′₁, which may be identical or different, represent a radical chosen from the groups:

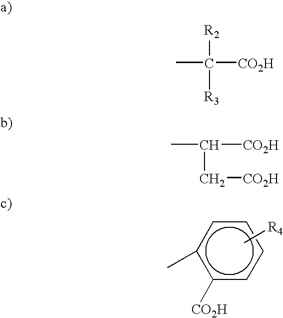

in which,

R₂ and R₃, which may be identical or different, denote a hydrogen atom or a linear or branched C₁-C₈ alkyl or C₂-C₈ alkenyl group; R₄ denotes a hydrogen atom or a linear or branched C₁-C₈ alkyl group, a linear or branched C₂-C₈ alkenyl group or a linear or branched C₁-C₈ alkoxy group;

R₅ and R₆ denote, independently of each other, a hydrogen atom, C₂-C₈ alkenyl, linear or branched C₁-C₈ alkoxy, phenyl, C₁-C₆ alkylcarboxylic or C₂-C₆ alkenylcarboxylic;

R₁₁ and R₁₂ have the same meanings as R₅ and R₆;

R₁₃ represents a linear or branched C₁-C₁₇ alkyl or C₂-C₁₇ alkenyl group or an acyl-CO—R₁₄ radical, with R₁₄ which denotes a linear or branched C₁-C₁₇ alkyl or C₂-C₁₇ alkenyl group;

R₁₅ is chosen from a hydrogen atom, a linear or branched C₁-C₈ alkyl or C₂-C₈ alkenyl group, a C₁-C₅ alkylcarboxylic or C₂-C₅ alkenylcarboxylic group, or a —COOH function;

R₂₁ and R′₂₁ denote groups of formulae:

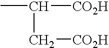

and —CH₂—CO₂H

R₂₂ represents a group as follows:

i) —CH₃

ii) —CO₂H

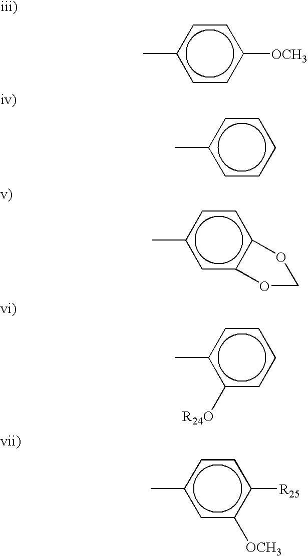

such that R₂₄ represents a group chosen from: —CH₂—COOH and —CO—CH₃ and R₂₅ represents a group chosen from: OH, OCH₃, OC(CH₃)₃, OCH₂COOH;

R₂₃ denotes a hydrogen atom, a linear or branched C₁-C₆ alkyl or C₂-C₆ alkenyl radical, or R₂₂ and R₂₃ together form an alkanediyl-(CH₂)m₃— group such that m₃ represents an integer ranging from 3 to 5, or R₂₂ and R₂₃ together form an aralkanediyl radical:

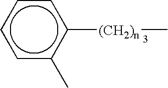

such that n₃ represents 3 or 4; or a salt thereof.

Preferably, the acids of the invention will be chosen from:

a) the compounds corresponding to formula (I) in which R₅=R₆=H, p₁=q₁ and R₁=R′₁;

b) the compounds corresponding to formula (II) in which r₂=1, R₁₃=H, R₁₅=—COOH and m=1 (cysteine derivatives) or m=2 (homocysteine derivatives);

c) the compounds corresponding to formula (III) in which R₂₂ is an aromatic radical, R₂₃=H and R₂₁=R′₂₁.

In particular, the acids of the invention are chosen from: methylene-2,2′-dithiodibenzoic acid, ethylene-2,2′-dithiodibenzoic acid, ethylene-2,2′-disulfinyldibenzoic acid, 2,2′-(1,3-propanediyldithio)dibenzoic acid, 2,2′-(1,4-butanediyldithio)dibenzoic acid, 2,2′-(1,5-pentanediyldithio)dibenzoic acid, 2,2′-(1,6-hexanediyldithio)dibenzoic acid, 2,2′-(1,6-hexanediyldisulfinyl)dibenzoic acid, α,α′-(1,6-hexanediyldithio)dibenzoic acid, 2,2′-(1,8-octanediyldithio)dibenzoic acid, 2,2′-(1,10-decanediyldithio)dibenzoic acid, α,α′-(1,10-decanediyldithio)dibenzoic acid, methylene-α,α′-dithiodiacetic acid, ethylene-α,α′-dithiodiacetic acid, ethylene-α,α′-disulfinyldiacetic acid, ethylene-α,α′-disulfonyldiacetic acid, α,α′-(1,6-hexanediyldithio)diacetic acid, α,α′-(1,6-hexanediyldisulfinyl)diacetic acid, α,α′-(1,6-hexanediyldisulfonyl)diacetic acid, α,α′-(1,10-decanediyldithio)diacetic acid, α,α′-(1,10-decanediyldisulfinyl)diacetic acid, α,α′-(1,10-decanediyldisulfonyl)diacetic acid, S-(carboxymethyl)cysteine, S-(carboxyethyl)cysteine, S-(4-carboxybutyl)cysteine, S-(carboxymethyl)sulfinyl cysteine, S-(carboxymethyl)sulfonyl cysteine, S-(1-carboxy-1-ethyl)cysteine, S-(2-carboxy-2-propyl)cysteine, α,α′-(p-methoxybenzylidenedithio)disuccinic acid, α,α′-(benzylidenedithio)disuccinic acid, α,α′-(piperonylidenedithio)disuccinic acid, α,α′-(vanillylidenedithio)disuccinic acid, α,α′-(veratrylidenedithio)disuccinic acid, α,α′-(4-butoxy-3-methoxybenzylidenedithio)disuccinic acid, α,α′-(4-carboxymethyloxy-3-methoxybenzylidenedithio)disuccinic acid, α,α′-(p-methoxybenzylidenedithio)diacetic acid, α,α′-(benzylidenedithio)diacetic acid, α,α′-(piperonylidenedithio)diacetic acid, α,α′-(vanillylidenedithio)diacetic acid, α,α′-(veratrylidenedithio)diacetic acid, and also the mono-salts and di-salts thereof with a mineral or organic base.

Preferably, S-carboxymethylcysteine and also the mono-salts and di-salts thereof with a mineral or organic base will be used according to the invention.

These compounds containing a thioether, sulfoxide or sulfone function are used as cosmetic agents for trapping toxic gases.

A subject of the present invention is also the use of compounds containing a thioether, sulfoxide or sulfone function, in or for the preparation of an antipollution cosmetic composition for topical application.

The cosmetic compositions used in the invention will advantageously contain from 0.001% to 10% and preferably between 0.01% and 5% by weight of compounds containing a thioether, sulfoxide or sulfone function relative to the total weight of the composition.

This composition may also contain at least one other antipollution compound.

Said antipollution compound may be chosen especially from anthocyans and/or derivatives thereof, ergothioneine and/or its derivatives, metal-chelating agents, for instance N,N′-dibenzylethylenediamine-N,N′-diacetic acid derivatives, antioxidants and cell extracts of plants from the Pontederiacea family. Among the antioxidants that will be chosen more particularly are polyphenols, for instance ellagic acid.

The cosmetic composition used in the invention may also contain a cosmetically acceptable medium, which more particularly consists of water and/or of a cosmetically acceptable organic solvent.

They may be chosen from the group consisting of hydrophilic organic solvents, amphiphilic solvents and lipophilic organic solvents, or mixtures thereof.

Among the hydrophilic organic solvents that may be mentioned, for example, are linear or branched lower monoalcohols containing from 1 to 8 carbon atoms, for instance ethanol, propanol, butanol, isopropanol and isobutanol, polyethylene glycols containing from 6 to 80 ethylene oxides, polyols such as propylene glycol, isoprene glycol, butylene glycol, glycerol, sorbitol, monoalkyl or dialkyl isosorbides, the alkyl groups of which contain from 1 to 5 carbon atoms, for instance dimethyl isosorbide, glycol ethers, for instance diethylene glycol monomethyl ether or monoethyl ether, and propylene glycol ethers, for instance dipropylene glycol methyl ether.

Amphiphilic organic solvents that may be mentioned include polyols such as propylene glycol (PPG) derivatives, such as esters of polypropylene glycol and of fatty acids, derivatives of PPG and of fatty alcohols, for instance PPG-23 oleyl ether, and PPG-36 oleate.

Lipophilic organic solvents that may be mentioned, for example, include fatty esters such as diisopropyl adipate, dioctyl adipate and alkyl benzoates.

The organic solvents are preferably chosen from monofunctional or polyfunctional alcohols, optionally oxyethylenated polyethylene glycols, polypropylene glycol esters, sorbitol and its derivatives, dialkyl isosorbides, glycol ethers and polypropylene glycol ethers, and fatty esters.

The organic solvents may represent from 5% to 98% of the total weight of the composition.

In order for the compositions used in the invention to be more pleasant to use, softer to apply, more nourishing and more emollient, it is possible to add a fatty phase to the medium of these compositions.

The fatty phase preferably represents from 0 to 50% relative to the total weight of the composition.

This fatty phase may comprise one or more oils preferably chosen from the group consisting of:

volatile or nonvolatile, linear, branched or cyclic, organomodified or non-organomodified, water-soluble or liposoluble silicones,

mineral oils such as liquid paraffin and liquid petroleum jelly,

oils of animal origin such as perhydrosqualene,

oils of plant origin such as sweet almond oil, avocado oil, castor oil, olive oil, jojoba oil, sesame oil, groundnut oil, macadamia oil, grape-seed oil, rapeseed oil or coconut oil,

synthetic oils such as purcellin oil and isoparaffins,

fluoro oils and perfluoro oils,

fatty acid esters such as purcellin oil.

Said fatty phase may also comprise as fatty substances one or more fatty alcohols, fatty acids or waxes (paraffin wax, polyethylene wax, carnauba wax or beeswax).

In a known manner, the compositions used in the invention may also contain adjuvants that are common in cosmetics, standard aqueous or lipophilic gelling agents and/or thickeners, hydrophilic or lipophilic active agents, preserving agents, antioxidants, fragrances, emulsifiers, moisturizers, pigmenting agents, depigmenting agents, keratolytic agents, vitamins, emollients, sequestering agents, surfactants, polymers, acidifying or basifying agents, fillers, free-radical scavengers, ceramides, sunscreens, especially ultraviolet screening agents, insect repellents, slimming agents, colorants, bactericides and antidandruff agents.

The amounts of these various adjuvants are those conventionally used in the fields under consideration.

Needless to say, a person skilled in the art will take care to select the optional compound(s) to be added to the composition according to the invention, such that the advantageous properties intrinsically associated with the composition in accordance with the invention are not, or are not substantially, adversely affected by the envisioned addition.

The compositions used according to the invention may be in any presentation form normally used for topical application, especially in the form of an aqueous, aqueous-alcoholic or oily solution, an oil-in-water or water-in-oil or multiple emulsion, an aqueous or oily gel, a liquid, pasty or solid anhydrous product or a dispersion of oil in an aqueous phase with the aid of spherules, these spherules possibly being polymer nanoparticles such as nanospheres and nanocapsules, or better still lipid vesicles of ionic and/or nonionic type.

The compositions used in the present invention may be more or less fluid and may have the appearance of a white or colored cream, an ointment, a milk, a lotion, a serum, a paste, a mousse or a solid.

They may optionally be applied to the skin in aerosol form.

They may also be applied in solid form, and for example in the form of a stick.

They may be used as care products and/or as makeup products.

Another subject of the invention consists of a cosmetic treatment process for protecting the body against the effects of pollution, which consists in applying to the skin a cosmetically effective amount of compounds containing a thioether, sulfoxide or sulfone function.

Another cosmetic treatment process according to the invention, for protecting the body against the effects of pollution, consists in applying to the skin a cosmetic composition according to the invention, as defined above.

The examples that follow are intended to illustrate the invention without, however, being limiting in nature.

### EXPERIMENTS

Principle

Ozone has the capacity to oxidize cell constituents, especially generating carbonylated proteins and lipid hydroperoxides. Quantification of the lipid hydroperoxides is one means of measuring the oxidative stress induced by exposing skin tissue to this pollutant. A decrease in their content indicates a protective effect.

Cell Type and Culturing

The study was performed on a monolayer culture of human keratinocytes. The cells are inoculated on D-3 into 48-well dishes at a rate of 25 000 cells/cm² in 500 μl of culture medium. The incubations are performed at 37° C., 5% CO₂ under a humid atmosphere.

Pretreatment of the Keratinocytes with S-carboxymethylcysteine (CMC)

The cells were pretreated for 24 hours with CMC (0.5 mg/ml, half-maximal dose that is nontoxic to the cells, dissolved in the culture medium).

Incorporation of an Oxidative Stress Marker, DCFH-DA (2,7-dichlorofluorescin diacetate)

Hydroperoxides constitute an intracellular stress marker. They are detected and quantified by means of a fluorescence technique (Lebel C. P., Ischiropoulos H. and Bondy S. C. (1992) Evaluation of the probe 2,7-dichlorofluorescin as an indicator of Reactive Oxygen Species formation and oxidative stress. Chem. Res. Toxicol.; 5: 227-231).

In the presence of intracellular hydroperoxides and peroxidases, DCFH is oxidized to fluorescent 2,7-dichlorofluorescein (DCF).

The cells, pretreated for 24 hours with CMC, are then washed with PBS buffer and placed in contact for 30 minutes with a solution of DCFH-DA (500 μl/well), prepared in the culture medium to a concentration of 320 μM.

Exposure to Ozone

The cells are again rinsed with PBS buffer and then placed in contact with a CMC solution at a concentration of 1 mg/ml in PBS (100 μl/well). They are then exposed to ozone (1 ppm), under a humid atmosphere, in an incubator set at 37° C.

Measurement of Ozone-induced Lipid Hydroperoxides

The formation of fluorescent DCF (excitation filter at 485 nm and emission filter at 530 nm) resulting from the production of hydroperoxides is measured after different times of exposure to ozone: 0, 5, 10 and 20 minutes.

Results

Toxicity of ozone toward human keratinocytes in culture, in the absence and presence of S-carboxymethylcysteine at a concentration of 200 μM, as a function of the exposure time (n=4).

Fluorescence observed in the presence of CMC, expressed
as % relative to the unprotected controls for each time

| 5 min. of contact | 10 min. of contact | 20 min. of contact |
|---|---|---|
| % fluorescence | % fluorescence | % fluorescence |
| observed ± | observed ± | observed ± |
| SEM | SEM | SEM |
| 32.4 ± 5.9 | 33.7 ± 5.0 | 46.5 ± 8.5 |

For each time, the fluorescence values of the unprotected controls are set at 100%. The results in the presence of CMC are then expressed relative to this control value. CMC significantly decreases the ozone-induced stress. This protection is at a maximum from 5 minutes of exposure onward (67.7% drop in induced stress). It is still significant after 20 minutes of exposure (53.5% drop in induced stress) relative to the unprotected controls.

Starting with a biological model, in vitro, using a cell of human epithelial origin (keratinocyte/skin), we have shown:

that a representative agent of a category of atmospheric pollutants such as ozone leads under our experimental conditions to the appearance of substantial oxidative stress,

that CMC exerts a highly significant protective effect against the effect induced by this pollutant.

### FORMULATION EXAMPLES

### Example 1

According to the usual preparation techniques, the constituents below are mixed together to prepare an emulsion.

COMPOSITION FOR TOPICAL APPLICATION

| | | | |
|---|---|---|---|
| S-carboxymethylcysteine | | 5 | g |
| polyethylene glycol oxyethylenated | | 3 | g |
| with 50 mol of ethylene oxide | | | |
| monodiglyceryl stearate | | 3 | g |
| liquid petroleum jelly | | 24 | g |
| cetyl alcohol | | 5 | g |
| water | qs | 100 | g |

### Example 2

In the same manner, an emulsion is prepared according to a standard technique, using the following compounds:

| | | | |
|---|---|---|---|
| S-carboxymethylcysteine | | 1 | g |
| octyl palmitate | | 10 | g |
| glyceryl isostearate | | 4 | g |
| purcellin oil | | 23 | g |
| vitamin E | | 1 | g |
| glycerol | | 3 | g |
| water | qs | 100 | g |

### Example 3

In the same manner, an emulsion is prepared according to a standard technique, using the following compounds:

| | | | |
|---|---|---|---|
| α,α′-(1,6-hexanediyldithio) diacetic acid | | 1 | g |
| octyl palmitate | | 10 | g |
| glyceryl isostearate | | 4 | g |
| liquid petroleum jelly | | 20 | g |
| sorbitol | | 2 | g |
| vitamin E | | 1 | g |
| glycerol | | 3 | g |
| water | qs | 100 | g |

### Example 4

In the same manner, an emulsion is prepared according to a standard technique, using the following compounds:

| | | | |
|---|---|---|---|
| α,α′-(1,10-decanediyldisulfinyl) diacetic acid | | 1 | g |
| octyl palmitate | | 10 | g |
| glyceryl isostearate | | 4 | g |
| liquid petroleum jelly | | 24 | g |
| vitamin E | | 1 | g |
| glycerol | | 3 | g |
| water | qs | 100 | g |

### Example 5

Starting with the constituents below, the following composition is formulated:

| | | | |
|---|---|---|---|
| α,α′-(p-methoxybenzylidenedithio) disuccinic acid | | 1.5 | g |
| jojoba oil | | 13 | g |
| methyl isopropyl para-benzoxybenzoate | | 0.05 | g |
| potassium sorbate | | 0.3 | g |
| cyclopentadimethylsiloxane | | 10 | g |
| stearyl alcohol | | 1 | g |
| stearic acid | | 4 | g |
| polyethylene glycol stearate | | 3 | g |
| vitamin E | | 1 | g |
| glycerol | | 3 | g |
| water | qs | 100 | g |

### Example 6

Starting with the constituents below, the following composition is formulated:

| | | | |
|---|---|---|---|
| α,α′-(p-methoxybenzylidenedithio) diacetic acid | | 1 | g |
| jojoba oil | | 13 | g |
| methyl isopropyl para-benzoxybenzoate | | 0.05 | g |
| potassium sorbate | | 0.3 | g |
| cyclopentadimethylsiloxane | | 10 | g |
| stearyl alcohol | | 1 | g |
| N,N′-bis (3-hydroxybenzyl) ethylenediamine-N,N′- | | 0.01 | g |
| diacetic acid | | | |
| stearic acid | | 4 | g |
| polyethylene glycol stearate | | 3 | g |
| vitamin E | | 1 | g |
| glycerol | | 3 | g |
| water | qs | 100 | g |

### Example 7

Starting with the constituents below, the following composition is formulated:

| | | | |
|---|---|---|---|
| α,α′-(vanillylidenedithio) diacetic acid | | 0.5 | g |
| jojoba oil | | 13 | g |
| methyl isopropyl para-benzoxybenzoate | | 0.05 | g |
| potassium sorbate | | 0.3 | g |
| cyclopentadimethylsiloxane | | 10 | g |
| stearyl alcohol | | 1 | g |
| stearic acid | | 4 | g |
| cell extract of water hyacinth | | 0.05 | g |
| polyethylene glycol stearate | | 3 | g |
| vitamin E | | 1 | g |
| glycerol | | 3 | g |
| water | qs | 100 | g |

The cell extract of water hyacinth from the Pontederiacea family (Eichhornia crassipes) was obtained by this process: 12 water hyacinth plants were washed with water and then crudely drained. After chopping in a knife mill (chopping processor), 700 g of ground material were obtained. Addition of 700 ml of H₂O and then 300 ml of MilliQ H₂O. Further chopping in the chopping processor for five minutes, centrifugation for 20 minutes at 8000×G, Whatmann GFD and then GFF filtration and freeze-drying: 5.43 g of lyophilizate are thus obtained.

1. The use in topical application, as an antipollution agent, preferably as an antipollution cosmetic agent, of at least one derivative containing a thioether, sulfoxide or sulfone function of formula (I), (II) or (III): 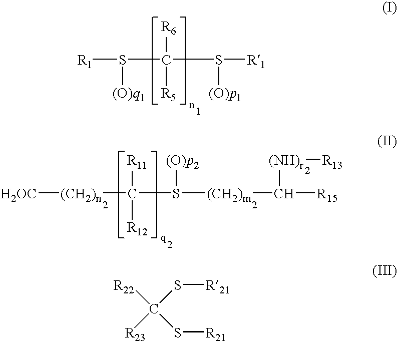
in which n₁ represents an integer ranging from 1 to 10, p₁ and q₁, which may be identical or different, represent 0, 1 or 2, n₂ represents an integer ranging from 0 to 5, p₂ represents 0, 1 or 2, q₂ represents 0, 1, 2 or 3, m₂ represents an integer ranging from 0 to 10, r₂ represents 0 or 1, q₂ and n₂ never both simultaneously being zero, R₁ and R′₁, which may be identical or different, represent a radical chosen from the groups: 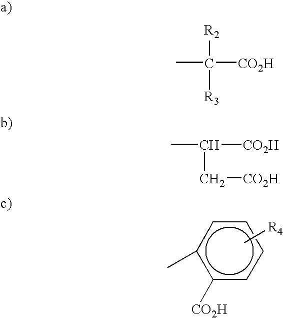
in which, R₂ and R₃, which may be identical or different, denote a hydrogen atom or a linear or branched C₁-C₈ alkyl or C₂-C₈ alkenyl group; R₄ denotes a hydrogen atom or a linear or branched C₁-C₈ alkyl group, a linear or branched C₂-C₈ alkenyl group or a linear or branched C₁-C₈ alkoxy group; R₅ and R₆ denote, independently of each other, a hydrogen atom, C₂-C₈ alkenyl, linear or branched C₁-C₈ alkoxy, phenyl, C₁-C₆ alkylcarboxylic or C₂-C₆ alkenylcarboxylic; R₁₁ and R₁₂ have the same meanings as R₅ and R₆; R₁₃ represents a linear or branched C₁-C₁₇ alkyl or C₂-C₁₇ alkenyl group or an acyl-CO—R₁₄ radical, with R₁₄ which denotes a linear or branched C₁-C₁₇ alkyl or C₂-C₁₇ alkenyl group; R₁₅ is chosen from a hydrogen atom, a linear or branched C₁-C₈ alkyl or C₂-C₈ alkenyl group, a C₁-C₅ alkylcarboxylic or C₂-C₅ alkenylcarboxylic group, or a —COOH function; R₂₁, and R′₂₁ denote groups of formulae: 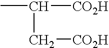
and —CH₂—CO₂H
R₂₂ represents a group as follows:
i) —CH₃
ii) —CO₂H 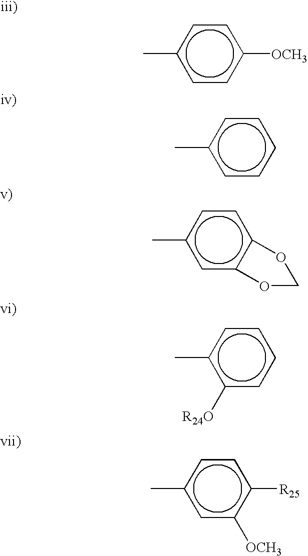
in which R₂₄ represents a group chosen from: —CH₂—COOH and —CO—CH₃ and R₂₅ represents a group chosen from: OH, OCH₃, OC(CH₃)₃, OCH₂COOH;
R₂₃ denotes a hydrogen atom, a linear or branched C₁-C₆ alkyl or C₂-C₆ alkenyl radical, or R₂₂ and R₂₃ together form an alkanediyl-(CH₂)m₃— group such that m₃ represents an integer ranging from 3 to 5, or R₂₂ and R₂₃ together form an aralkanediyl radical: 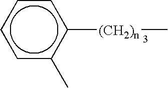
such that n₃ represents 3 or 4; or a salt thereof.

2. The use as claimed in claim 1, characterized in that the derivative containing a thioether function corresponds to formula (I) in which R₅=R₆=H, p₁=q₁ and R₁=R′₁.

3. The use as claimed in claim 1, characterized in that the derivative containing a sulfoxide function corresponds to formula (II) in which r₂=1, R₁₃=H, R₁₅=—COOH and m=1 (cysteine derivatives) or m=2 (homocysteine derivatives).

4. The use as claimed in claim 1, characterized in that the derivative containing a sulfone function corresponds to formula (III) in which R₂₂ is an aromatic radical, R₂₃=H and R₂₁=R′₂₁.

5. The use as claimed in any one of claims 1 to 4, characterized in that the derivative containing a thioether, sulfoxide or sulfone function is chosen from methylene-2,2′-dithiodibenzoic acid, ethylene-2,2′-dithiodibenzoic acid, ethylene-2,2′-disulfinyldibenzoic acid, 2,2′-(1,3-propanediyldithio)dibenzoic acid, 2,2′-(1,4-butanediyldithio)dibenzoic acid, 2,2′-(1,5-pentanediyldithio)dibenzoic acid, 2,2′-(1,6-hexanediyldithio)dibenzoic acid, 2,2′-(1,6-hexanediyldisulfinyl)dibenzoic acid, α,α′-(1,6-hexanediyldithio)dibenzoic acid, 2,2′-(1,8-octanediyldithio)dibenzoic acid, 2,2′-(1,10-decanediyldithio)dibenzoic acid, α,α′-(1,10-decanediyldithio)dibenzoic acid, methylene-α,α′-dithiodiacetic acid, ethylene-α,α′-dithiodiacetic acid, ethylene-α,α′-disulfinyldiacetic acid, ethylene-α,α′-disulfonyldiacetic acid, α,α′-(1,6-hexanediyldithio)diacetic acid, α,α′-(1,6-hexanediyldisulfinyl)diacetic acid, α,α′-(1,6-hexanediyldisulfonyl)diacetic acid, α,α′-(1,10-decanediyldithio)diacetic acid, α,α′-(1,10-decanediyldisulfinyl)diacetic acid, α,α′-(1,10-decanediyldisulfonyl)diacetic acid, S-(carboxymethyl)cysteine, S-(carboxyethyl)cysteine, S-(4-carboxybutyl)cysteine, S-(carboxymethyl)sulfinyl cysteine, S-(carboxymethyl)sulfonyl cysteine, S-(1-carboxy-1-ethyl)cysteine, S-(2-carboxy-2-propyl)cysteine, α,α′-(p-methoxybenzylidenedithio)disuccinic acid, α,α′-(benzylidenedithio)disuccinic acid, α,α′-(piperonylidenedithio)disuccinic acid, α,α′-(vanillylidenedithio)disuccinic acid, α,α′-(veratrylidenedithio)disuccinic acid, α,α′-(4-butoxy-3-methoxybenzylidenedithio)disuccinic acid, α,α′-(4-carboxymethyloxy-3-methoxybenzylidenedithio)disuccinic acid, α,α′-(p-methoxybenzylidenedithio)diacetic acid, α,α′-(benzylidenedithio)diacetic acid, α,α′-(piperonylidenedithio)diacetic acid, α,α′-(vanillylidenedithio)diacetic acid, α,α′-(veratrylidenedithio)diacetic acid, and also the mono-salts and di-salts thereof with a mineral or organic base.

6. The use as claimed in any one of claims 1 to 5, characterized in that the derivative containing a thioether, sulfoxide or sulfone function of formula (I), (II) or (III) is chosen from carboxymethylcysteine and also the mono-salts and di-salts thereof with a mineral or organic base.

7. The use in topical application of at least one derivative containing a thioether, sulfoxide or sulfone function of formula (I), (II) or (III) as defined in one of claims 1 to 6, as a cosmetic agent for trapping toxic gases such as ozone.

8. The use in topical application of at least one derivative containing a thioether, sulfoxide or sulfone function of formula (I), (II) or (III) as defined in claim 1 or 6, to increase cell regeneration and a return to homeostasis in keratin materials, in order to obtain healthier keratin materials.

9. The use of a derivative containing a thioether, sulfoxide or sulfone function of formula (I), (II) or (III) as defined in any one of claims 1 to 6, in or for the preparation of an antipollution composition, preferably an antipollution cosmetic composition, for topical application.

10. The use as claimed in claim 9, characterized in that said antipollution cosmetic composition contains from 0.005% to 10% and preferably from 0.1% to 5% by weight of compounds containing a thioether, sulfoxide or sulfone function of formula (I), (II) or (III) relative to the total weight of the composition.

11. The use as claimed in claim 9 or 10, characterized in that said composition also contains at least one other antipollution compound.

12. The use as claimed in claim 11, characterized in that said antipollution compound is chosen from anthocyans and/or derivatives thereof, ergothioneine and/or its derivatives, metal-chelating agents, for instance N,N′-dibenzylethylenediamine-N,N′-diacetic acid derivatives, antioxidants, for instance ellagic acid, and cell extracts of plants from the Pontederiacea family.

13. The use as claimed in any one of claims 9 to 12, characterized in that the composition also contains a cosmetically acceptable medium consisting of water and/or of at least one organic solvent chosen from the group consisting of hydrophilic organic solvents, lipophilic organic solvents and amphiphilic solvents, or mixtures thereof.

14. The use as claimed in claim 13, characterized in that the organic solvents are chosen from the group consisting of monofunctional or polyfunctional alcohols, optionally oxyethylenated polyethylene glycols, polypropylene glycol esters, sorbitol and its derivatives, dialkyl isosorbides, glycol ethers and polypropylene glycol ethers, and fatty esters.

15. The use as claimed in claim 13 or 14, characterized in that the organic solvent(s) represent(s) from 5% to 98% relative to the total weight of the composition.

16. The use as claimed in any one of claims 9 to 15, characterized in that the composition also comprises at least one fatty phase.

17. The use as claimed in claim 16, characterized in that the fatty phase represents from 0 to 50% relative to the total weight of the composition.

18. The use as claimed in any one of claims 9 to 17, characterized in that the composition also contains at least one additive chosen from the group consisting of standard aqueous or lipophilic gelling agents and/or thickeners, hydrophilic or lipophilic active agents, preserving agents, antioxidants, fragrances, emulsifiers, moisturizers, pigmenting agents, depigmenting agents, keratolytic agents, vitamins, emollients, sequestering agents, surfactants, polymers, acidifying or basifying agents, fillers, free-radical scavengers, ceramides, sunscreens, especially ultraviolet screening agents, insect repellents, slimming agents, colorants, bactericides and antidandruff agents.

19. The use as claimed in any one of claims 9 to 18, characterized in that the composition is in the form of an aqueous, aqueous-alcoholic or oily solution, an oil-in-water or water-in-oil or multiple emulsion, an aqueous or oily gel, a liquid, pasty or solid anhydrous product or a dispersion of oil in an aqueous phase with the aid of spherules.

20. The use as claimed in any one of claims 9 to 19, characterized in that the composition has the appearance of a white or colored cream, an ointment, a milk, a lotion, a serum, a paste, a mousse or a solid.

21. The use as claimed in any one of claims 9 to 20, characterized in that the composition has a pH of between 3 and 8.

22. A cosmetic treatment process for protecting the body against the effects of pollution, characterized in that it consists in applying to keratin material a cosmetically effective amount of at least one derivative containing a thioether, sulfoxide or sulfone function of formula (I), (II) or (III) as defined in one of claims 1 to 6.

23. A cosmetic treatment process for protecting the body against the effects of pollution, characterized in that it consists in applying to keratin material a cosmetic composition as defined in any one of claims 9 to 21.
